(19) United States
(12) Patent Application Publication
(10) Pub. No.: US 2017/0299548 A1
(43) Pub. Date: Oct. 19, 2017

(54) Nucleic Acid Delivery Controlling System and Method for Manufacturing Same, and Nucleic Acid Sequencing Device

(71) Applicant: Hitachi High-Technologies Corporation, Minato-ku, Tokyo (JP)

(72) Inventors: Hiroshi YOSHIDA, Tokyo (JP); Rena AKAHORI, Tokyo (JP); Takanobu HAGA, Tokyo (JP)

(73) Assignee: Hitachi High-Technologies Corporation, Minato-ku, Tokyo (JP)

(21) Appl. No.: 15/517,333

(22) PCT Filed: Oct. 14, 2015
(86) PCT No.: PCT/JP2015/079000

§ 371 Date: Apr. 6, 2017

(30) Foreign Application Priority Data
Oct. 24, 2014 (JP) 2014-217124

(51) Int. Cl. G01N 27/447; C12Q 1/68

(52) U.S. Cl. CPC G01N 27/44791; C12Q 1/6869

(57) ABSTRACT

The present invention provides: a nucleic acid delivery controlling system in which a novel delay principle is utilized to greatly delay the nanopore passing rate of a nucleic acid strand, thereby enabling the stable analysis of a nucleotide sequence; a method for manufacturing the nucleic acid delivery controlling system; and a nucleic acid sequencing device. The present invention relates to a nucleic acid delivery controlling system, equipped with a passage through which a nucleic acid strand can pass, said nucleic acid delivery controlling system being characterized in that the passage through which a nucleic acid strand can pass has at least one nanochannel having multiple passages per one nanopore through which only one molecule of the nucleic acid strand can pass, the nanochannel has a microphase-separated structure composed of a block copolymer that is composed of a hydrophobic polymer chain and a hydrophilic polymer chain, and the nanochannel contains the hydrophilic polymer chain of the block copolymer as the main component.

### TECHNICAL FIELD

The present invention relates to a device for controlling transport of a nucleic acid strand, and to a method for manufacturing the device. The present invention also relates to a nucleic acid sequencing apparatus for reading the nucleotide sequence of a nucleic acid strand.

### BACKGROUND ART

Passing a single molecule of biopolymer through a pore of about sub-nanometer to several nanometer size (hereinafter, referred to as “nanopore”) embedded in a thin membrane of a thickness measuring about several angstrom to several tens of nanometers causes a change in the pattern of physical properties, electrical and/or optical, near the nanopore in a manner than depends on the sequence pattern of monomers in the biopolymer. When the biopolymer is a nucleic acid such as deoxyribonucleic acid (DNA) and ribonucleic acid (RNA), a pattern change occurs according to the nucleotide sequence of the nucleic acid.

There have been active studies of methods that take advantage of this phenomenon for analysis of biopolymer monomer sequence, specifically a sequence analysis of DNA nucleotide sequence for biopolymer DNA, or, more concisely, DNA sequencing. In these methods, a nanopore is often used with an electrolyte-containing solution placed on the both sides of a thin membrane. A voltage is applied across the thin membrane to create a potential difference, and pass the electrolyte-containing solution through the nanopore.

The DNA strand sequencing technique that currently holds the most promise focuses attention on the electrical ion current produced under applied voltage. This technique works under the principle that different monomers create characteristic changes in the magnitude of ion current observed upon translocation of a DNA strand through the nanopore. Aside from the ion current method, a technique is widely known that uses a tunnel current that passes between a pair of electrodes formed at a nanopore portion. The principle behind this technique is that the amount of the tunnel current observed upon translocation of a biopolymer through the nanopore varies from monomer to monomer.

Both of these techniques are capable of directly reading a biopolymer without requiring the traditional chemical procedures that involve fragmentation of a biopolymer. The techniques are available as a next-generation DNA nucleotide sequence analysis system in the case of a DNA biopolymer, and an amino acid sequence analysis system in the case of a protein biopolymer. These systems are expected to enable reading much longer sequence lengths than conventionally achieved. The following descriptions are based on a DNA biopolymer.

Two types of nanopore devices are available: a biopore using a protein embedded in a lipid bilayer membrane and having a center pore, and a solid pore formed through an insulating thin membrane formed by a semiconductor process.

The biopore uses a pore (a diameter of 1.2 nm, and a thickness of 0.6 nm) of an altered protein (for example, Mycobacterium smegmatis porin A (MspA)) embedded in a lipid bilayer membrane, and measures a change in the amount of ion current by using the pore as a DNA sequence detector. However, the measured change in the amount of ion current contains information from different bases when the pore thickness is larger than the single base unit (the distance between the adjacent monomer bases of DNA is 0.34 nm). Another drawback in addition to the lack of space resolution is that the device, because it uses a protein, deteriorates as the pore portion of the protein denatures in a manner that depends on solution conditions or environmental conditions. This is problematic in terms of stability and lifetime, or robustness of the device.

In the solid pore, a nanopore can be formed through a thin membrane of a single molecule layer such as graphene and molybdenum disulfide. The thickness is sufficient for providing a space resolution sufficient to read a single base unit. Further, unlike protein, the material is stable under various solution conditions and environmental conditions, and the device is advantageous in terms of robustness. Another advantage is that parallel nanopore portions can be fabricated using a semiconductor process. Because of these advantages, the solid pore has attracted interest as a device superior to the biopore.

The method that electrophoreses a DNA strand by directly using the ion current-generating potential difference as a driving force is the most common means of transporting a DNA strand to regions near a nanopore, and passing the DNA strand through it. However, since the electrophoresed DNA strand passes through the nanopore at very high speeds, the method produces a signal value that contains signals from the adjacent bases. A technique that slows the translocation speed is thus required to enable a sequence analysis. Specifically, a translocation speed of 0.01 to 1 μs/base is currently achieved while it needs to be desirably 100 μs/base or slower. To achieve this, the translocation speed needs to be slowed by a factor of at least about 100 to 10,000. It would be possible to obtain a single-base signal if the translocation speed could be reduced to such low speeds.

Various techniques have been proposed to achieve this. There are studies of methods that adjust physical properties of a solution. For example, there is a method for slowing translocation speed through a nanopore whereby high-concentration glycerol is added to increase solution viscosity, and thus the frictional force that acts in the opposite direction from the force that pulls a DNA strand in electrophoresis (NPL 1). There is also a study of a method for slowing translocation speed through a nanopore whereby lithium ions are added to solution to reduce the apparent negative charge of a DNA strand, and decrease the force that pulls the DNA strand in electrophoresis (NPL 2).

Aside from the methods that adjust the physical properties of a solution, methods that make changes in the device are also studied. For example, there are studies of a method that makes changes to the nanopore itself. In a known simple method, the diameter of a nanopore is made smaller to increase the frictional force against the translocation of a DNA strand through the nanopore, and to thereby slow the translocation speed through the nanopore (NPL 3).

Methods that provide a new structure for a device are also studied. PTL 1 discloses a method in which two-dimensional obstacles are installed in a nanopore device configured from two-dimensional channels. This publication discloses a structure in which a group of nanosize obstacles (e.g., columns) is orderly arranged with a distance on the both sides of a thin membrane that has been processed to include a nanopore.

Gel materials configured from polymers, resins, inorganic porous materials, or beads are given as other examples of the obstacles. It is mentioned that the electrophoresed biopolymer collides with the obstacles, and creates a frictional force that acts against the direction of electrophoresis to reduce the translocation speed through the nanopore.

NPL 4 discloses other means of achieving obstacles, specifically a structure in which a group of random layers of resin nanowires is provided on the upstream side of a nanopore. The frictional force that occurs as the electrophoresed biopolymer collides with the nanowires is used to slow the translocation speed through the nanopore.

### CITATION LIST

### Patent Literature

### PTL 1: JP-A-2014-074599

### Non Patent Literature

### NPL 1: D. Fologea, et al., Nano Lett., 2005, Vol. 5(9), p. 1734.

### NPL 2: S. W. Kowalczyk, et al., Nano Lett., 2012, Vol. 12(2), p. 1038.

### NPL 3: R, Akahori, et al., Nanotechnology, 2014, Vol. 25, p. 275501.

NPL 4: A. H. Squires, et al., J. Am. Chem. Soc., 2013, Vol. 135(44), p. 16304.

### SUMMARY OF INVENTION

### Technical Problem

A problem of the traditional methods is the insufficient slowing effect. For example, the translocation time is slowed by a factor of only about 5 by addition of glycerol in the method that uses double-stranded DNA as the subject biopolymer, and that adds glycerol or other materials to adjust solution properties such as viscosity. Another drawback is that the additives are passed with the DNA strand. The difference between single-base signal values from different bases is accordingly small, and detection of different bases is difficult. The method that uses single-stranded DNA, and adds lithium ions reduces speed by a factor of only about 10 after addition of lithium ions. For example, the speed is reduced by a factor of only about 15 in the traditional method that slows the translocation speed of a double-stranded DNA biopolymer through a nanopore with the use of an obstacle.

All of the known methods thus fail to sufficiently reduce the translocation speed of a nucleic acid strand such as a DNA strand through a nanopore to speeds that enable nucleotide sequence analysis, and there is a need for development of some other means.

The present invention was accomplished in view of the foregoing problems. The present invention is intended to provide a nucleic acid transport controlling device that, through the use of a novel slowing principle, greatly slews the translocation speed of a nucleic acid strand through a nanopore, and enables a stable nucleotide sequence analysis. The invention is also intended to provide a method for manufacturing the device, and a nucleic acid sequencing apparatus.

### Solution to Problem

The present inventors conducted intensive studies, and found that a nanochannel with closely packed hydrophilic polymer chains can be formed through self-assembly of a block copolymer of a hydrophobic polymer chain and a hydrophilic polymer chain. The present inventors also found that the transport speed of a nucleic acid strand can be greatly reduced by translocation of a nucleic acid strand in such a nanochannel. The present inventors thought of using the nanochannel for a nucleic acid transport controlling device having a nanopore.

In an aspect, a nucleic acid transport controlling device of the present invention includes a nucleic acid strand translocation pathway,

wherein the nucleic acid strand translocation pathway includes one or more multipath nanochannels per nanopore that allows passage of only one molecule of nucleic acid strand,

wherein the nanochannels have a microphase-separated structure of a block copolymer of a hydrophobic polymer chain and a hydrophilic polymer chain, and

wherein the nanochannels contain the hydrophilic polymer chain of the block copolymer as a main component.

In another aspect, a nucleic acid transport controlling device of the present invention includes a nucleic acid strand translocation pathway,

wherein the nucleic acid strand translocation pathway includes one or more multipath nanochannels per nanopore that allows passage of only one molecule of nucleic acid strand,

wherein the nucleic acid transport controlling device includes an insulating base material having one or more of the nanopore, and a thin membrane directly or indirectly disposed above the insulating base material,

wherein the thin membrane includes one or more of the nanochannels, and a matrix disposed around the nanochannels, and

wherein the nanochannels are packed with a hydrophilic polymer chain immobilized at the interface between the nanochannels and the matrix.

### Advantageous Effects of Invention

The nucleic acid transport controlling device according to the present invention can reduce the transport speed of a nucleic acid strand to speeds that enable reading a nucleotide sequence. The nucleic acid transport controlling device according to the present invention can be produced by a simple method. The present invention is therefore highly useful for the production of an accurate and reliable nucleic acid sequencing apparatus.

Other objects, configurations, and advantages of the present invention will be more clearly understood from the descriptions of the embodiments below.

This specification incorporates the substance of the specification and/or drawings of Japanese Patent Application No. 2014-217124 on which the present patent application is based.

### BRIEF DESCRIPTION OF DRAWINGS

FIG. 1 is a schematic view representing a cross sectional structure of a nucleic acid sequencing apparatus using a nucleic acid transport controlling device 10 of the present invention.

FIG. 2 is a schematic diagram representing a random channel structure and an upright cylindrical structure as exemplary structures of the block copolymer thin membrane 20.

FIG. 3 is an enlarged view schematically representing the upright cylindrical structure as an example of the constituting unit of the block copolymer thin membrane 20.

FIG. 4 is a schematic view representing a random channel structure and an upright cylindrical structure as exemplary nanochannel structures.

FIG. 5 shows a scanning transmission electron micrograph of a PEO-b-PMA(Az) thin membrane having a random, channel structure, and a scanning electron micrograph of a PEO-b-PMA(Az) thin membrane having an upright cylindrical structure.

FIG. 6 shows schematic views of cross sectional structures of various configurations of nucleic acid transport controlling devices of Examples and Comparative Examples.

FIG. 7 shows a scanning transmission electron micrograph of a PEO-b-PMA(Az) thin membrane having a random channel structure in the vicinity of an aperture portion of a nucleic acid transport controlling device, and a scanning transmission electron micrograph of a PEO-b-PMA (Az) thin membrane having an upright cylindrical structure in the vicinity of an aperture portion of a nucleic acid transport controlling device.

FIG. 8 is a plot representing the result of a high-resolution measurement of time-course changes in the amount of ion current observed for a buffer solution sample containing a ssPolyA chain in a nucleic acid transport controlling device of Example having a first configuration.

FIG. 9 is a diagram representing a distribution of translocation times of a ssPolyA chain in a nucleic acid transport controlling device of Example having the first configuration.

### DESCRIPTION OF EMBODIMENTS

Preferred embodiments of the present invention are described below in detail. The embodiments of the invention are described by appropriately using the accompanying drawings. The following descriptions represent specific examples of the substance of the present invention. The present invention is not limited to the descriptions below, and various changes and modifications may be made thereto by a skilled artisan within the scope of the technical ideas disclosed herein. In all drawings describing the present invention, the same reference numerals are used to refer to members having the same functions, and descriptions of such members may not be repeated.

### Nucleic Acid Sequencing Apparatus

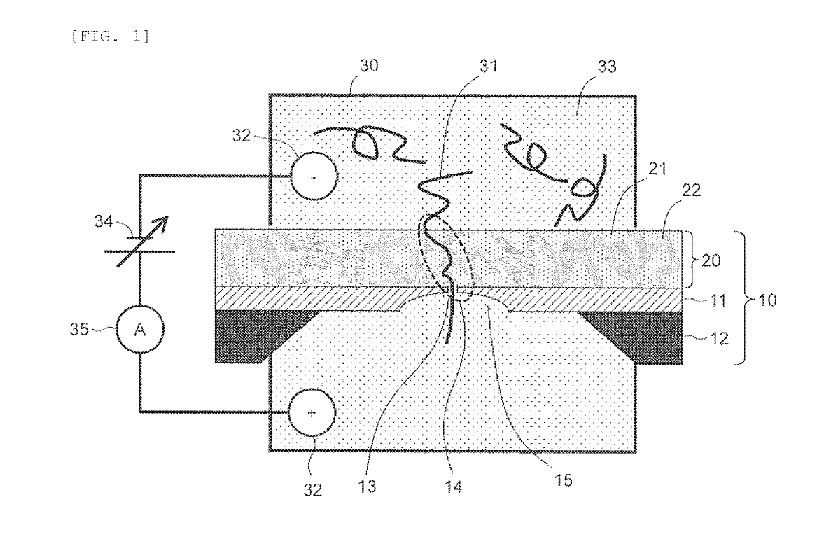

FIG. 1 is a schematic view representing an example of a cross sectional structure of a nucleic acid sequencing apparatus using a nucleic acid transport controlling device of the present invention. The nucleic acid sequencing apparatus of the present invention includes a nucleic acid transport controlling device 10, two solution cells 30 that are in communication with each other via a nucleic acid strand translocation pathway 14 of the nucleic acid transport controlling device 10, and an electrode 32 provided for each of the two solution cells 30 to apply voltage between the solution cells 30. The solution cells 30 contain an electrolyte aqueous solution 33, and are in communication with each other via the translocation pathway 14 of the nucleic acid transport controlling device 10. One of the solution cells 30 contains a nucleic acid strand 31, a sample for which the sequence is to be read. The nucleic acid strand translocation pathway 14 of the nucleic acid transport controlling device 10 has a nanopore 13, and a nanochannel 22. The electrodes 32 are installed in the solution cells 30, and a voltage is applied to the electrodes to pass the nucleic acid strand 31 through the translocation pathway 14 in the nucleic acid transport controlling device 10. FIG. 1 represents an embodiment with the nucleic acid transport controlling device 10 in which a single nucleic acid strand translocation pathway 14 is disposed. However, the number of nucleic acid strand translocation pathways in the nucleic acid transport controlling device is not particularly limited in the nucleic acid sequencing apparatus of the present invention. For example, in another embodiment, the nucleic acid transport controlling device 10 may have a plurality of nucleic acid strand translocation pathways 14 that are disposed parallel to each other. In FIG. 1, the region of translocation pathway 14 indicated by a dotted line is shown to illustrate the function of the translocation pathway 14. The range of the nanochannel 22 where the nucleic acid strand is passed is not limited to the region shown in the diagram.

As used herein, “nucleic acid” means deoxyribonucleic acid (DNA) or ribonucleic acid (RNA). The nucleic acid is preferably a single-stranded nucleic acid strand, more preferably a single-stranded DNA strand. The nucleotide sequence of the nucleic acid strand can be read with high accuracy by applying the present invention to the nucleic acid.

In an embodiment in which the nucleotide sequence of a nucleic acid strand is determined by measuring the value of the ion current that passes through the translocation pathway during the translocation of the nucleic acid strand 31 through the translocation pathway 14, time-course changes of the current amount between the electrodes 32 may be measured with an ammeter 35. A sensor is therefore not particularly required in this embodiment. The ammeter 35 is desirably a device capable of measuring weak current at high time resolution and low noise level.

In an embodiment in which a sensor for determining the nucleic acid strand type is used to read the nucleotide sequence of the nucleic acid strand 31 passing through the translocation pathway 11 of the nucleic acid transport controlling device 10, the sensor is installed on both sides the nucleic acid transport controlling device 10, or inside the nucleic acid transport controlling device 10. In FIG. 1, the sensor is omitted for simplification. The means of reading the nucleotide sequence of the nucleic acid strand, and the configuration of the sensor used for such means are not particularly limited. There are many reports of means for measuring physical quantities such as changes in the tunnel current that traverses the nucleic acid strand, and amounts of charge on nucleic acid strands. Any of these known means may be used in the embodiment of the present invention. Alternatively, the chemical composition of the nucleic acid strand passing through the translocation pathway 14 may be spectroscopically measured using, for example, raman spectroscopy, or infrared absorption. When using spectroscopic means, it is preferable to use an excitation method based on a localized enhanced optical field such as a plasmon, in order to obtain a space resolution that corresponds to the base size.

### Nucleic Acid Transport Controlling Device

The nucleic acid transport controlling device 10 of the present invention has the nucleic acid strand translocation pathway 14. In the nucleic acid strand translocation pathway 14, one or more nanochannels 22 having a plurality of paths are provided per nanopore 13 that allows passage of only a single molecule of nucleic acid strand. For a single nanopore 13 that allows passage of only a single molecule of nucleic acid strand, the nucleic acid strand translocation pathway 14 has preferably one or two, particularly one nanochannel 22 having a plurality of paths. Preferably, the nanopore 13 and the nanochannel 22 are disposed in contact with each other, or by being separated from each other. In an embodiment in which the nanopore 13 and the nanochannel 22 are spaced apart from each other, a nucleic acid strand aligning portion may be disposed between the nanopore 13 and the nanochannel 22 so as to surround the nanochannel-side aperture of the nanopore 13. The nucleic acid strand aligning portion is a space, or a layer of an arbitrarily chosen material. When the nucleic acid strand aligning portion is disposed, a plurality of nucleic acid strands can be aligned, and only one molecule of nucleic acid strand can be channeled to a single nanopore 13 even when more than one nucleic acid strand passes through the multiple paths of the nanochannel 22, as will be described later.

In an aspect of the present invention, the nanochannel 22 has a microphase-separated structure of a block copolymer of a hydrophobic polymer chain and a hydrophilic polymer chain. In this case, the nanochannel 22 contains the hydrophilic polymer chain of the block copolymer as a main component. In another aspect of the present invention, the nanochannel 22 is packed with hydrophilic polymer chains immobilized at the interface between the nanochannel 22 and a matrix 21.

The nanochannel 22 may be configured from a single domain having a channel structure, or may be configured as an assembly of a plurality of such domains. In an embodiment in which the nanochannel is configured as an assembly of a plurality of domains, a single domain having a channel structure is also referred to as “nanochannel unit” in this specification.

In the nucleic acid strand translocation pathway of the present invention, one or more nanochannels corresponding to a single nanopore each have a plurality of paths that allows passage of the nucleic acid strand and electrolyte ions. The nucleic acid strand translocation pathway of the present invention having this feature is advantageous in terms of accurately reading the nucleotide sequence of the nucleic acid strand using a blocked current method. Referring to FIG. 1, applying a voltage to the nucleic acid transport controlling device 10 of the present invention immersed in an aqueous solution of an electrolyte such as potassium chloride creates an ion current flow as the electrolyte passes through the nanopore. Here, when the electrolyte aqueous solution contains the nucleic acid strand 31, a translocation event of the nucleic acid strand 31 through the nanopore 13 occurs. The ion current value continuously varies according to the types of the bases forming the nucleic acid strand 31 passing through the nanopore 13. The blocked current method is a means of reading the nucleotide sequence of a nucleic acid strand using the amount of change of the ion current. The blocked current method is desirable because it does not require a sensor for reading the nucleotide sequence of a nucleic acid strand.

In order to read the nucleotide sequence of a nucleic acid strand using the blocked current method, a sufficient amount of ion current needs to be passed both stably and constantly to the nanopore that passes the nucleic acid strand. In the nucleic acid transport controlling device of the present invention, one or more multipath nanochannels are provided per nanopore. This feature enables providing the ion current amount and the stability needed to read the nucleotide sequence of the nucleic acid strand. For example, when one molecule of nucleic acid strand is passed through the nanochannel of the nucleic acid strand pathway, the nucleic acid strand passes through one of the paths of the nanochannel. The electrolyte ions, on the other hand, can pass through one or more of the nanochannel paths, excluding the translocation path of the nucleic acid strand. In this way, a stable ion current flow can be achieved when passing one molecule of nucleic acid strand through the nucleic acid strand pathway. The nucleic acid strand translocation pathway of the present invention can thus exhibit only the effect of slowing the nucleic acid strand translocation speed, without affecting the behavior of the passing electrolyte ions, specifically, for example, the resistance against electrolyte ions.

In an aspect of the present invention, the nucleic acid transport controlling device 10 of the present invention may include a base material 11 having one or more nanopores 13, and a thin membrane 20 directly or indirectly disposed above the base material 11. In this case, the thin membrane 20 includes one or more nanochannels 22, and the matrix 21 disposed around the nanochannels 22. As used herein, “thin membrane 20 being disposed above the base material 11” includes not only when the thin membrane 20 is disposed on the upper surface of the base material 11 as used, but when the thin membrane 20 is disposed on the lower surface, or on the both surfaces of the base material 11. When the thin membrane 20 is disposed on the both surfaces of the base material 11, it is preferable that the both thin membranes 20 include one or more nanochannels 22, and the matrix 21 surrounding the nanochannels 22. In such an embodiment, two nanochannels having a plurality of paths may be provided per nanopore 13 that allows passage of only one molecule of nucleic acid strand in the nucleic acid strand translocation pathway 14. As used herein, “thin membrane 20 being directly disposed above the base material 11” means that the base material 11 and the thin membrane 20 are in contact with each other, and “thin membrane 20 being indirectly disposed above the base material 11” means that the base material 11 and the thin membrane 20 are disposed with a space in between, either partially or as a whole. In an embodiment in which the thin membrane 20 is indirectly disposed above the base material 11, specifically when the base material 11 and the thin membrane 20 are disposed with a space in between, the nucleic acid strand aligning portion may be disposed between the base material 11 and the thin membrane 20 so as to surround the thin membrane-side aperture of the nanopore 13.

The shape of the nanochannel 22 is not limited to the randomly branched, interconnected structure shown in FIG. 1. The nanochannel 22 may have, for example, a structure with an assembly formed by one or more arranged cylindrical or lamellar nanochannel units 23 that are disposed through the thin membrane 20. The nanochannel 22 may have a branched structure. When the nanochannel 22 has a branched structure, the nanochannel 22 typically forms a continuous, orderly structure with the surrounding matrix 21. The nanochannel structure will be described in later sections.

The nanopore 13 has diameter D. The diameter D may be appropriately selected according to the molecule passed through the nanopore. For example, when the molecule passed through the nanopore is a single-stranded nucleic acid, the diameter D is preferably 0.7 nm or more, more preferably 0.9 nm or more. The diameter D is preferably 5 nm or less, more preferably 1.5 nm or less. The diameter D is preferably 0.7 to 5 nm, more preferably 0.9 to 1.5 nm. A single-stranded nucleic acid molecule can pass through the nanopore when the diameter D has the foregoing lower limits. With a diameter D having the foregoing upper limits, the passage through the nanopore can be limited to only one molecule of single-stranded nucleic acid.

The nanopore 13 may be circular (for example, a true circle, or elliptical) or polygonal in shape, or may have any other shape created by distorting these shapes. Preferably, the nanopore 13 is circular in shape. When the shape of the nanopore 13 is not a true circle, the diameter D of the nanopore 13 is the diameter of an inscribed true circle in a cross section of the nanopore 13 at the surface of the base material 11.

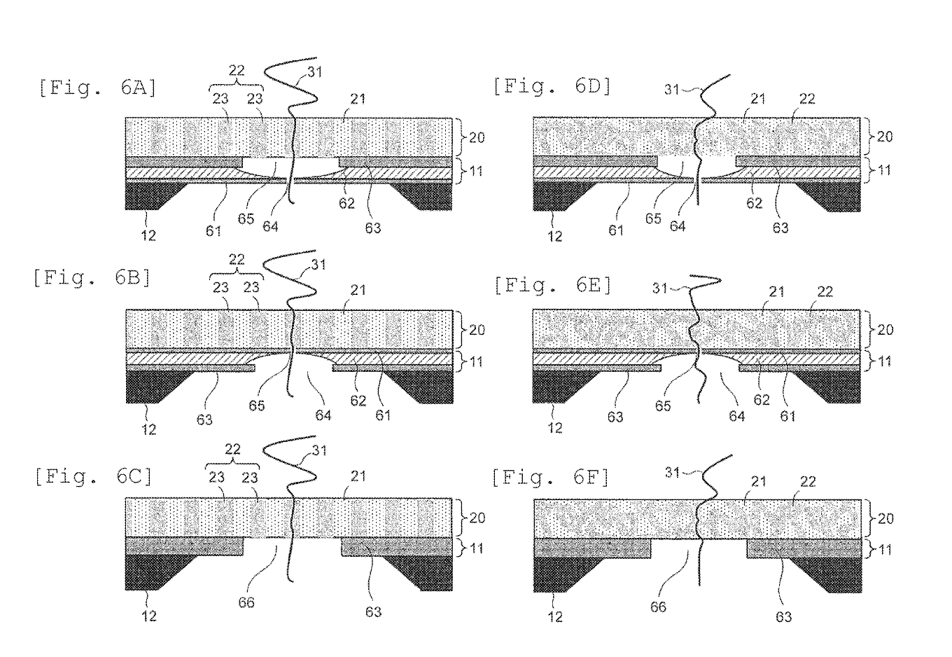

As shown in FIG. 1, the base material 11 may have a monolayer structure formed of a single layer, or, as shown in FIG. 6, a multilayer structure formed of more than one layer. An embodiment in which the base material 11 has a multilayer structure is particularly advantageous because such a base material can be fabricated with ease from a layer having a nanopore and of a thickness corresponding to the size of a single base, and a layer having other functions (for example, a layer having the nucleic acid strand aligning portion).

The base material 11 is typically insulating. The material of the base material 11 is not particularly limited, as long as the nanopore 13 can be formed. The material of the base material 11 is preferably, for example, silicon nitride (SiN, for example, Si₃N₄), silicon oxide (SiO₂), hafnium oxide (HfO₂), or graphene. When produced from these materials, the base material 11 can have corrosion resistance against the electrolyte solution 33, and the nanopore 13 can be formed with ease. When a blocked current value is used to read the nucleotide sequence of the nucleic acid strand, the material of the base material 11 is preferably a sheet-like two-dimensional material of one-atom thickness, such as SiN, and graphene. The base material 11 formed of such a two-dimensional material can have a thickness that corresponds to the size of a single base. For example, when the base material 11 has a monolayer structure, the base material 11 is produced preferably with a two-dimensional material such as above. When the base material 11 has a multilayer structure, the plurality of layers may be produced using the same material selected from the foregoing materials, or may be produced using different materials selected from the foregoing materials. In this case, it is preferable that the layer having the nanopore 13 is fabricated from a two-dimensional material such as above. When the layer having the nanopore 13 is produced using a two-dimensional material, the base material can have a thickness that corresponds to the size of a single base in a region around the nanopore 13. The nucleotide sequence of the nucleic acid strand can be read with high accuracy with such a configuration.

The base material 11 has a thickness of preferably 100 nm or less, particularly 50 nm or less so as to form the fine nanopore 13 of the desired diameter D. For sufficient strength, the base material 11 has a thickness of preferably 10 nm or more. When a blocked current value is used to read the nucleotide sequence of the nucleic acid strand, the base material 11 has a thickness of preferably 0.3 nm or more. The thickness corresponds to the size of a single base. The nucleotide sequence of the nucleic acid strand can be read with high accuracy when the thickness has the foregoing lower limit.

The base material 11 may have the foregoing thickness throughout the base material 11. However, the thickness of the base material 11 may be different in a region around the nanopore 13, and in other regions. In this case, the base material 11 preferably has a multilayer structure. For example, when a blocked current value is used to read the nucleotide sequence of the nucleic acid strand, the nanopore layer of the base material 11 has a thickness of preferably 0.3 to 2.0 nm, and the base material 11 having the multilayer structure has a total thickness of 10 to 100 nm. The base material 11 can have regions of different thicknesses with such a configuration.

The base material 11 may have a base pore 15. The base pore 15 is joined to the nanopore 13 at one end of the whole aperture portion, or at the smallest part of the aperture portion. Specifically, one end of the base pore 15 has an aperture portion of diameter D joined to the nanopore 13, and the other end of the base pore 15 has an aperture portion of diameter D′. Preferably, the base pore 15 is disposed in the base material 11 having a multilayer structure. For example, in the embodiment in which the base material 11 has a multilayer structure, the base pore is disposed preferably in layers 62 and 63 disposed above or below a layer 61 having a nanopore, as shown in FIG. 6(a), (d), and (e). With such a configuration, the nanopore and the base pore can be formed in different layers. In the base pore 15, the diameter D′ is preferably 0.7 nm or more, more preferably 0.9 nm or more. The diameter D′ is preferably 100 nm or less, more preferably 50 nm. or less. The diameter D′ is preferably 0.7 to 100 nm, more preferably 0.9 to 50 nm. When the diameter D′ of the base pore 15 has the foregoing lower limits, the base pore 15 can be joined to the nanopore 13 at one end. When the diameter D′ of the base pore 15 has the foregoing upper limits, a base material can be used that has a thickness of the desired range (described later) in a region around the nanopore 13, and a thicker thickness in other regions.

Preferably, the base pore is disposed in the upper surface of the base material as used. In this way, the base pore will be disposed between the nanopore formed in the base material, and the nanochannel, and functions as the nucleic acid strand aligning portion. In this case, the base pore serving as the nucleic acid strand aligning portion will be in communication with the plurality of nanochannel units constituting the nanochannel, and joins the paths of the nanochannel units to the nanopore.

The base material 11 may be used by itself. However, in order to improve the hardness or ease of handling of the base material 11, it is preferable to dispose a support substrate 12 below the base material 11, as shown in FIG. 1. Preferably, the support substrate 12 is disposed on the lower surface of the base material 11 as used. In this case, the support substrate 12 is disposed preferably in contact with a part of the lower surface of the base material 11, more preferably around the aperture portion of the nanopore 13 and/or the base pore 15 on the surface of the base material 11. With the support substrate 12 disposed in this fashion, the hardness or ease of handling of the base material 11 can be improved while maintaining the nucleic acid strand translocation pathway.

The surface of the base material 11 may be chemically altered to improve the compatibility between the base material 11 surface and the thin membrane 20. This may be achieved by grafting a polymer chain on the surface of the base material 11, or through reaction of a coupling agent with the surface of the base material 11. Alternatively, a surface improving technique, such as a plasma treatment or a UV treatment, may be applied to the surface of the base material 11.

The base material 11 may be produced according to a known method, for example, the method disclosed in JP-A-8-248198. For example, the base material 11 (for example, a silicon nitride or silicon oxide film) is formed on a surface of the support substrate 12 (for example, a silicon wafer), and a part of the support substrate 12 is removed by anisotropic etching using, for example, a tetramethylammonium hydroxide (TMAH) solution or a potassium hydroxide (KOH) aqueous solution. When the base material 11 has a multilayer structure, the desired cross sectional shape may be produced using a known method, for example, a combination of photolithography and etching, widely used in the field of, for example, semiconductor fabrication.

A variety of known semiconductor processing techniques may be used for the formation of the nanopore 13. The method used for the process of forming the nanopore 13 may be appropriately selected, taking into account the size (diameter D) of the nanopore 13, and/or process time. For example, it is possible to use a process using a focused ion beam (FIB) that uses a particle beam such as gallium ions and helium ions, a process using a focused electron beam (EB), or a photolithography process. When forming a single nanopore 13, a direct process such as a FIB and EB process is preferred. When producing a device capable of parallel reading with an array of nanopores 13, a photolithography process is preferred because of a shorter processing time.

Alternatively, the nanopore 13 also may be formed by using the dielectric breakdown phenomenon, whereby a nucleic acid transport controlling device 10 with no nanopore 13 is installed in solution cells 30, and a pulsed voltage is applied to the electrodes 32 with the nucleic acid transport controlling device 10 immersed in the electrolyte (for example, H. Kwok et al. PLoS ONE 9 (3), 2014). The method of the present embodiment is desirable in that the size (diameter D) of the nanopore 13 can be adjusted while measuring the amount of current passing between the electrodes.

### Block Copolymer Thin Membrane

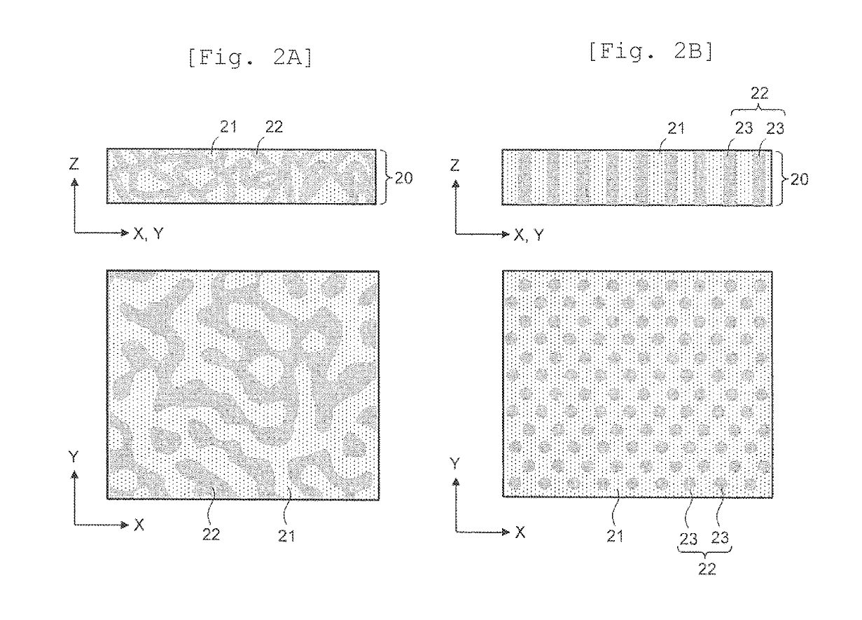

In an aspect of the present invention, the thin membrane contains a block copolymer. In this specification, the thin membrane containing a block copolymer is also referred to as a “thin membrane of a block copolymer”, or a “block copolymer thin membrane”. The block copolymer thin membrane 20 includes one or more nanochannels 22, and the matrix 21 (continuous phase) surrounding the nanochannels 22. The nanochannel 22 has a microphase-separated structure of a block copolymer of a hydrophobic polymer chain and a hydrophilic polymer chain. FIG. 2 is a schematic diagram representing an embodiment showing the microphase-separated structure of the block copolymer thin membrane 20, in which (a) shows a nanochannel having a random branched structure (hereinafter, also referred to as “random channel structure”), and (b) shows a nanochannel having an upright cylindrical structure vertically aligned through the thin membrane (hereinafter, also referred to simply as “cylindrical structure”).

In the embodiment in which the nanochannel 22 has a random channel structure, the nanochannel 22 has an interconnected continuous structure in the block copolymer thin membrane 20. The matrix 21 also has an interconnected continuous structure. In this case, the nanochannel 22 and the matrix 21 have a complementary continuous structure. In this specification, the complementary continuous structure of nanochannel and matrix is also referred to as “co-continuous structure”. The embodiment in which the nanochannel and the matrix have a co-continuous structure includes, for example, not only the random channel structure shown in FIG. 2(a), but a gilloidal structure with an orderly branched structure. In the present invention, the embodiment in which the nanochannel and the matrix have a co-continuous structure may employ either structure.

In the embodiment in which the nanochannel 22 has a cylindrical structure, the nanochannel units 23 having a cylindrical structure are arranged in the matrix 21 in such an orientation that the nanochannel units 23 penetrate through the block copolymer thin membrane 20. The nanochannel 22 having a cylindrical structure forms a pattern in which the nanochannel units 23 having a cylindrical structure are orderly arranged in a hexagonal close-packed structure on the horizontal surface (i.e., the upper surface or lower surface) of the block copolymer thin membrane 20 as used. The embodiment in which the nanochannel has a structure with an assembly of independently arranged nanochannel units includes, for example, not only the cylindrical structure shown in FIG. 2(b), but a structure in which lamellar nanochannel units are arranged in such an orientation that the nanochannel units penetrate through the block copolymer thin membrane 20. In the present invention, the embodiment in which the nanochannel has a cylindrical structure may employ either structure.

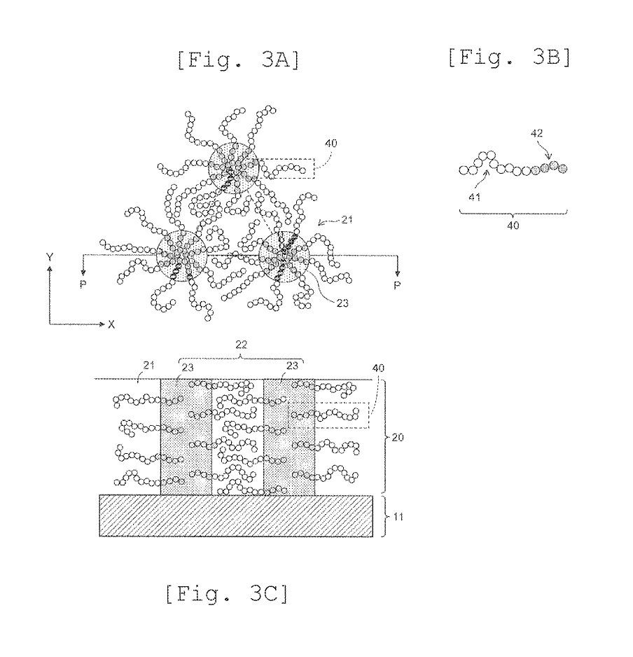

The microphase-separated structure of the block copolymer is described below with reference to FIG. 3. FIG. 3 is an enlarged view schematically illustrating the constituting unit of the block copolymer thin membrane 20, taking as an example the nanochannel units 23 constituting the nanochannel having a cylindrical structure. The block copolymer thin membrane 20 contains the block copolymer 40 either as the sole component or a main component. When the block copolymer 40 is an amphiphatic diblock copolymer of a hydrophobic polymer chain 41 and a hydrophilic polymer chain 42, a molecule of the block copolymer 40 has a chemical structure in which the hydrophobic polymer chain 41 and the hydrophilic polymer chain 42 are bound to each other at their terminals, as shown in FIG. 3(b). The block copolymer 40 may be an AB diblock copolymer in which the hydrophobic polymer chain 41 and the hydrophilic polymer chain 42 are linked to each other at the terminals, or an ABA triblock copolymer. The block copolymer 40 may be an ABC block copolymer of three or more polymer chains with an additional third polymer chain. Aside from the linear block copolymers in which the polymer chains are linked in series, the block copolymer 40 may be a star block copolymer in which the polymer chains are linked to each other at a single point. These structures also fall within the embodiment of the block copolymer of the present invention.

The block copolymer may be synthesized using a suitable method. For improved regularity of the microphase-separated structure, it is preferable to produce the block copolymer using a synthesis method that makes the molecular weight distribution as small as possible, for example, such as living polymerization, and atom-transfer radical-polymerization (ATRP).

Examples of the hydrophilic polymer chain 42 as a constituting unit of the block copolymer 40 include polymer chains containing polyethylene oxide (PEO), polylactic acid (PLA), polyhydroxyalkylmethacrylate (for example, polyhydroxyethylmethacrylate (PHEMA)), polyacrylamide (for example, N,N-dimethylacrylamide), or ionic polymers (for example, a polymer of unsaturated carboxylic acids such as polyacrylic acid, and polyacrylmethacrylic acid; polyamino acids, nucleic acids, or salts thereof). The hydrophilic polymer chain 42 is preferably polyethylene oxide, polylactic acid, or polyhydroxyethylmethacrylate, more preferably polyethylene oxide.

Examples of the hydrophobic polymer chain 41 as a constituting unit of the block copolymer 40 include polymer chains containing polystyrene (PS), polyalkylmethacrylate (for example, polymethylmethacrylate (PMMA)), polyvinylpyridine, polyalkylsiloxane (for example, polydimethylsiloxane), or polyalkyldiene (for example, polybutadiene). Preferably, the hydrophobic polymer chain 41 is one in which the main chain formed by any of the foregoing polymer chains has a liquid-crystalline side chain containing a mesogenic group that exhibits a liquid crystalline property. Examples of such mesogenic groups include groups having an azobenzene, stilbene, benzylidene aniline, biphenyl, naphthalene, or cyclohexane skeleton. The liquid-crystalline side chain containing the mesogenic group may be joined to the main chain via a spacer group, as required. In this case, the spacer group joined to the mesogenic group may be, for example, an alkyl group, an alkoxy group, or an alkoxyalkyl group. Th e spacer group is preferably linear. The spacer group has preferably 4 or more carbon atoms, more preferably 5 or more carbon atoms, further preferably 8 or more carbon atoms, particularly preferably 10 or more carbon atoms. Examples of the hydrophobic polymer chain 41 having the side chain include polymer chains having a structure in which the alkyl moiety of polyalkylmetnacrylate is substituted with the liquid-crystalline polymer chain either partially or completely. In the block copolymer, polyethylene oxide is particularly preferred as the hydrophilic polymer chain 42 combined with the hydrophobic polymer chain 41 having the liquid-crystalline side chain. With the liquid-crystalline side chain introduced to the hydrophobic polymer chain of the block copolymer, the block copolymer can easily form the microphase-separated structure through self-assembly, and form the nanochannel. In the embodiment in which the nucleic acid transport controlling device of the present invention has the block copolymer thin membrane 20, introducing the liquid-crystalline side chain to the hydrophobic polymer chain of the block copolymer enables the block copolymer to easily form the microphase-separated structure through self-assembly. This makes it possible to form the nanochannel 22 of a structure penetrating through the block copolymer thin membrane 20 from the upper surface to the lower surface as used.

In the liquid-crystalline block copolymer containing the hydrophobic polymer chain 41 having the liquid-crystalline side chain, the matrix 21 with the main-component hydrophobic polymer chain 41 having the liquid-crystalline side chain develops a liquid crystal phase. In the embodiment in which the nucleic acid transport controlling device of the present invention has the block copolymer thin membrane 20, the liquid-crystalline side chain homeotropically aligns itself with respect to the upper surface (free surface) of the block copolymer thin membrane 20 as used upon the matrix 21 developing a liquid crystal phase. With this alignment effect, the nanochannel 22 turns upright with respect to the upper and lower surfaces of the block copolymer thin membrane 20 as used, and easily aligns itself in a direction that penetrates through the thin membrane. The orientation of the nanochannel 22 often varies with factors such as the thickness of the block copolymer thin membrane 20, the process temperature during self-assembly, and/or the surface state of the base material. These may pose difficulties in the orientational control of the nanochannel 22. In the present invention, because the liquid-crystalline block copolymer is used, the nanochannel can be aligned in a direction that penetrates through the block copolymer thin membrane.

The microphase-separated structure of the block copolymer formed by self-assembly of the block copolymer can be specified by the composition ratio of the constituting unit block, for example, by the ratio of volumes occupied by the polymer chains representing the constituting unit of the block copolymer. The microphase-separated structure of the block copolymer, specifically the nanochannel structure changes from a lamellar (plate-like) structure to a co-continuous gyroid structure, and to a cylindrical structure and a spherical structure as the block composition ratio of the block copolymer increases in a range of 0.5 to 1.0. A nanochannel of the desired structure can thus be obtained by appropriately deciding the composition ratio of the hydrophobic polymer chain and the hydrophilic polymer chain.

### Slowing Transport Speed of Nucleic Acid Strand

In an aspect of the present invention, the nanochannel 22 contains the hydrophilic polymer chain as a main component, as shown in FIG. 3. In another aspect of the present invention, the hydrophilic polymer chain fills inside of the nanochannel 22. After intensive studies, the present inventors found that a nucleic acid strand passes through the nanochannel 22 immersed in an aqueous solution, and that the translocation speed of the nucleic acid strand is much slower than in a micropore not packed with the hydrophilic polymer chain, or in a bulk water-soluble polymer gel. The present invention was completed on the basis of these findings.

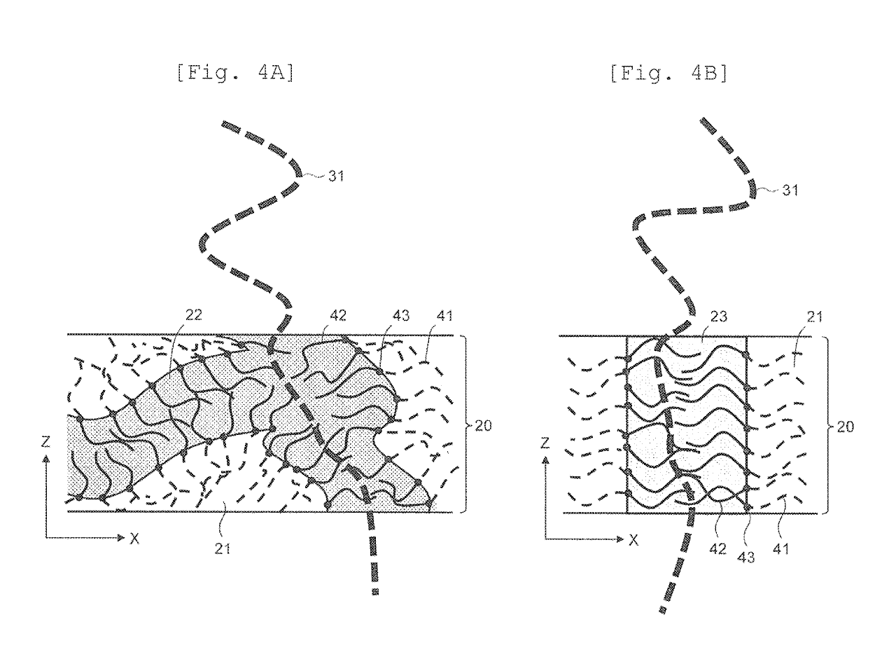

The effect of the present invention that slows the transport of a nucleic acid strand is described below with reference to FIG. 4. FIG. 4(a) is an enlarged view schematically showing a part of the nanochannel with the random channel structure, and FIG. 4(b) is an enlarged view schematically showing a part of the channel unit 23 constituting the nanochannel having the upright cylindrical structure. In an aspect of the present invention, the nanochannel 22 contains the hydrophilic polymer chain 42 as a main component. In another aspect of the present invention, the nanochannel 22 is packed with the hydrophilic polymer chain 42. Preferably, the matrix (hereinafter also referred to as “hydrophobic matrix”) 21 containing the hydrophobic polymer chain 41 as a main component is disposed around the nanochannel 22. In this case, the hydrophilic polymer chain 42 and the hydrophobic polymer chain 41 have a linkage point 43 of a structure immobilized at the interface between the nanochannel 22 and the hydrophobic matrix 21 (for example, at the side surface of the nanochannel 22).

Here, the density of the hydrophilic polymer chain 42 in the nanochannel 22 in a dry state is considered to be substantially the same as the density in a solid state. When the nanochannel 22 having such a structure is immersed in an aqueous solution, small molecules such as the water and the electrolyte contained in the aqueous solution diffuse into the hydrophilic nanochannel 22. However, the hydrophilic polymer chain 42 does not greatly swell because it is immobilized to the side surface of the nanochannel 22 at the linkage point 43. Accordingly, the density of the hydrophilic polymer chain 42 in the nanochannel 22 does not greatly decrease even after the nanochannel 22 is immersed in the aqueous solution. It is envisaged that this creates a fine space inside the nanochannel 22 filled with an ultrahigh-density gel.

It was completely unknown whether translocation of a high-molecular-weight nucleic acid strand would occur upon entry in such a fine space. The present inventors conducted intensive studies, and found that translocation of the nucleic acid strand 31 through the nanochannel 22 occurs when a potential difference is created between the terminal aperture portions of the nanochannel 22.

The transport speed of the nucleic acid strand 31 can be controlled by appropriately adjusting, for example, the diameter of the nanochannel 22 when the nanochannel 22 has the random channel structure. When the nanochannel 22 has the cylindrical structure, the transport speed of the nucleic acid strand 31 can be controlled by appropriately adjusting, for example, the diameter of the nanochannel unit 23, the path length of the nanochannel 22 over which translocation of the nucleic acid strand 31 occurs, and/or the density of the main component hydrophilic polymer chain 42 of the nanochannel 22. In the embodiment in which the nucleic acid transport controlling device of the present invention has the block copolymer thin membrane 20, the path length of the nanochannel 22 has a correlation with the thickness of the block copolymer thin membrane 20. For stable translocation of the nucleic acid strand 31 and for sufficient slowing effect, the block copolymer thin membrane 20 should have a thickness of preferably 10 nm or more, particularly 20 nm or more, and preferably 500 nm or less, particularly 100 nm or less.

### Block Copolymer Thin Membrane Producing Method

In the embodiment in which the nucleic acid transport controlling device of the present invention has the block copolymer thin membrane 20, the block copolymer thin membrane 20 having the nanochannel 22 can be produced using a method that includes the following steps.

First, the nanopore 13 is formed in the base material 11. This step may be performed by using the method described above. The nanopore forming step may be performed before or after the steps described below. Preferably, the nanopore forming step is performed after the step of forming the nanochannel to be described later. By performing these steps in this order, the nucleic acid transport controlling device of the present invention can be produced with ease, without requiring the process of aligning the terminal aperture of the nanochannel with the nanopore.

The block copolymer 40 of the predetermined chemical structure and composition is synthesized by polymerization reaction. The polymerization reaction is preferably living polymerization or atom-transfer radical-polymerization (ATRP) because it allows controlling the molecular weight, the composition, and/or the molecular weight distribution of the block copolymer 40, as described above. The shape and size of the nanochannel 22, and/or the distance between the domains vary according to the molecular weight of the block copolymer 40, and the molecular weight ratio of the hydrophobic polymer chain 41 and the hydrophilic polymer chain 42 representing the constituting unit of the block copolymer. The nanochannel of the desired structure can thus be obtained by appropriately adjusting the reaction conditions of the polymerization reaction.

The block copolymer 40 produced is dissolved in a solvent, and the resulting block copolymer solution is used to form the block copolymer thin membrane 20 above the base material 11, preferably on the upper surface of the base material 11 as used. The solvent is not particularly limited, as long as it can uniformly dissolve the block copolymer. The solvent may be selected from various organic solvents commonly used in the art, for example, such as toluene, and chloroform. Because the block copolymer 40 is typically amphiphatic, a solvent that can uniformly dissolve the block copolymer may not be available depending on the chemical composition of the polymer chains combined. In such a case, a mixed solvent of different solvents may be used as a solvent for dissolving the block copolymer 40.

Known means such as spin coating and dip coating may be appropriately used for the formation of the block copolymer thin membrane 20. The block copolymer thin membrane 20 of the desired thickness can be obtained by appropriately adjusting conditions such as the concentration of a block copolymer solution, the type of solvent, rotation speed (in the case of spin coating), and/or pulling rate (in the case of dip coating) so that the block copolymer thin membrane 20 has the predetermined thickness.

The block copolymer molecule 40 inside the block copolymer thin membrane 20 formed in the foregoing step exists in a state before completion of the self-assembly micropnase separation process as the evaporation of the solvent stops the process. Typically, the phase separation rapidly progresses when the dissimilar constituting unit polymer chains of the block copolymer have a large repulsive force (strong segregation) as in the amphipnatic block copolymer used in the present invention. In such a block copolymer, the microphase separation progresses to some extent even after the solvent has evaporated. In this case, a random channel structure, or a random branched structure to be more specific, often forms inside the block copolymer. The nanochannel 22 can thus be formed using the foregoing principle in the embodiment in which the nanochannel 22 has a random channel structure.

In the embodiment in which the nanochannel 22 has an orderly structure, for example, a lamellar structure, a gyroid structure, or a cylindrical structure, formed by a transition of the block copolymer to a stable equilibrium state, the microphase separation process can progress through self-assembly of the block copolymer by annealing the block copolymer thin membrane 20 formed on the base material 11. As used herein, the term, “annealing” means a process by which the block copolymer 40 is maintained in a freely movable state inside the block copolymer thin membrane 20 to form a structure that minimizes the free energy of the thin membrane. Annealing may be performed using known methods, for example, a process that heats the block copolymer 40 to at least the glass transition point of the constituting unit polymer chain (heat annealing), or a process that swells the block copolymer thin membrane 20 through exposure to a steam of solvent (solvent annealing).

In the embodiment that performs heat annealing with a liquid-crystalline block copolymer, the transition temperature of the liquid crystal also needs to be carefully considered. In the liquid-crystalline block copolymer, the liquid-crystalline side chain develops a liquid crystal property when the isotropic phase, which is randomly dispersed at a temperature equal to or greater than the liquid crystal transition temperature, is aligned in a certain direction in temperatures less than the liquid crystal transition temperature. When using the liquid-crystalline block copolymer, a uniform microphase-separated structure can thus be obtained by cooling the block copolymer to a temperature below the liquid crystal transition temperature after heating the block copolymer to a temperature equal to or greater than the liquid crystal transition temperature. For example, when using a block copolymer 40 of the hydrophobic polymer chain 41 having a liquid-crystalline side chain that includes an azobenzene skeleton as a mesogenic group, and the hydrophilic polymer chain 42 of polyethylene oxide (PEO), it is preferable to anneal the block copolymer 40 by heating it to the liquid crystal transition temperature of 100° C. or higher temperatures, and then cooling the block copolymer 40 to 90° C., a temperature below the liquid crystal transition temperature and not less than the glass transition point.

### EXAMPLES

The present invention is described below in greater detail using Examples. It is to be noted that the following Examples are not intended to limit the technical scope of the present invention.

### Production Example 1: Production of Nucleic Acid Transport Controlling Device Having Nanochannel with Upright Cylindrical Structure

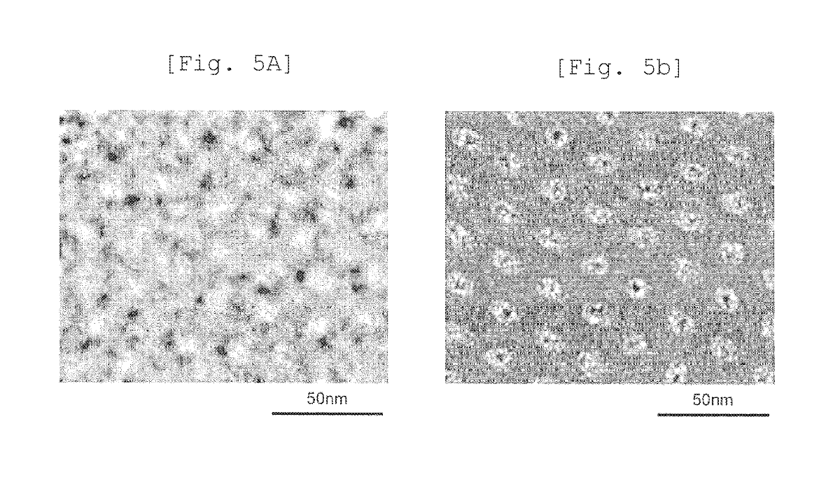

In this Production Example, Example of the nucleic acid transport controlling device of the present invention using the nanochannel having the upright cylindrical structure will be described with reference to FIG. 5 to FIG. 9, along with corresponding Comparative Examples.

### (1) Synthesis of Liquid-Crystalline Block Copolymer, and Evaluation of Physicochemical Properties

The block copolymer used is PEO-b-PMA(Az) comprised of a polyethylene oxide (PEO) hydrophilic polymer chain, and a hydrophobic polymer chain for which a polymethacrylate derivative (PMA(Az)) having a liquid-crystalline side chain with an azobenzene mesogenic group was used. The chemical formula of the block copolymer is as follows.

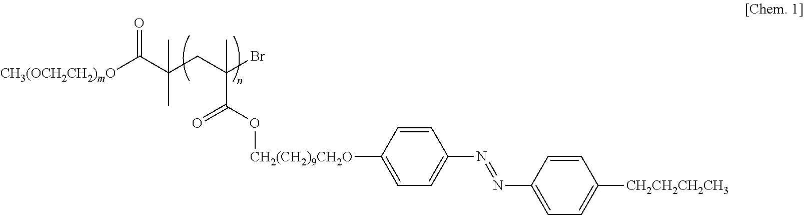

In the formula, m and n are natural numbers representing the degrees of polymerization of PEO and PMA(Az), respectively.

In this Production Example, the block copolymer had m=114, and n=34.

PEO-b-PMA (Az) was polymerized by atom-transfer radical-polymerization according to the method described in Y. Tian et al., Macromolecules 2002, 35, 3739-3747, The degree of polymerization of the resulting block copolymer was determined by ¹H NMR and GPC.

The block copolymer (PEO₁₁₄-b-PMA(Az)₃₄) was evaluated for self-assembly structure. First, PEO₁₁₄-b-PNLA (Az)₃₄ was dissolved in toluene in a concentration of 1.5 weight %. The resulting solution was spin coated on a SiN thin membrane surface in a thickness of about 50 nm to fabricate two as-spun (a state after spin coating) samples. The thickness was adjusted by varying the rotation speed of spin coating. The desired thickness was obtained by performing the spin coating process at a rotation speed of about 3,000 rpm.

One of the as-spun samples was charged into a vacuum oven, and heat annealed using the method described below. The heat annealing caused the PEO₁₁₄-b-PMA(Az)₃₄ thin membrane to self-assemble, and form a microphase-separated structure of the block copolymer. First, the as-spun sample was left unattended for 1 hour under 140° C. heated conditions in a vacuum. At this temperature, formation of an isotropic phase by PMA(Az)₃₄ was confirmed by separately performed polarization microscope observation. The heated sample was then cooled to 90° C. to allow a phase transition in PMA(Az)₃₄ from isotropic phase to smectic liquid phase. The cooled sample was allowed to stand in this state for 3 hours, and this was followed by natural cooling. The heat annealing completed the self-assembly of the block copolymer.

The structures of the as-spun sample and the neat annealed sample were observed under a scanning transmission electron microscope (STEM; HD-2700 available from Hitachi High-Technologies). STEM observation was performed after staining the PEO phase by exposing the sample to a steam, of ruthenium (Ru). FIG. 5 shows examples of STEM micrographs.

FIG. 5(a) shows an STEM dark-field image of the as-spun PEO₁₁₄-b-PMA(Az)₃₄ thin membrane. FIG. 5(b) shows an STEM dark-field image of the heat annealed PEO₁₁₄-b-PMA(Az)₃₄ thin membrane. Ruthenium selectively stains PEO. Accordingly, the PEO phase appears lighter, and the PMA(Az) phase appears darker in STEM dark-field image.

As can be seen in FIG. 5(a), the PEO₁₁₄-b-PMA(Az)₃₄ thin membrane was shown to have a random channel structure of randomly joined, branched PEO nanochannels in an as-spun state. The nanochannel diameter was about 10 nm. As can be seen in FIG. 5(b), the annealed PEO₁₁₄-b-PMA(Az)₃₄ thin membrane was shown to have an upright cylindrical structure in which the independent cylindrical PEO nanochannel units (hereinafter, also referred to as “PEO cylinders”) were hexagonally arranged upright with respect to the membrane. The PEO cylinder diameter was 9 nm, and the interval between the centers of the cylinders was 23 nm.

### (2) Fabrication of Nucleic Acid Transport Controlling Device

In this Production Example, nucleic acid transport controlling devices of three different configurations were fabricated, as schematically represented in the cross sectional structures shown in FIG. 6(a) to (c). The first configuration shown in FIG. 6(a) represents Example of the nucleic acid transport controlling device of the present invention. The second and third configurations shown in FIG. 6(b) and FIG. 6(c) represent Comparative Examples for the first configuration of Example.

First, a device substrate was prepared by depositing a base material 11 on the upper surface of a Si wafer provided as the support substrate 12. In order to form aperture portions of different shapes by a combination of photolithography and etching processes, a multilayer film of a sandwich structure including SiN layers 61 and 63 disposed on the upper and lower surfaces, respectively, of a SiO₂ layer 62 was used as the base material 11.

In the first configuration of Example shown in FIG. 6(a), the upper aperture 65 of the base pore formed in the upper surface of the base material 11 had a diameter of 50 nm, and the lower aperture 64 of the base material 11 that is in communication with the upper aperture 65 had a diameter of 2.5 nm. In this configuration, the lower aperture 64 serves as an aperture portion for the nanopore. The upper aperture 65 serves as an aperture portion for the base pore. The base pore having the upper aperture 65 serves as a nucleic acid strand aligning portion by which the number of nanochannel units 23 constituting the nanochannel 22 connected to the nanopore (the number of independent PEO cylinders in this example) is limited within a predetermined range.

In the second configuration of Comparative Example shown in FIG. 6(b), the upper aperture 65 formed in the upper surface of the base material 11 had a diameter of 2.5 nm, and the lower aperture 64 in communication with the upper aperture 65 of the base material 11 had a diameter of 50 nm. In this configuration, the upper aperture 65 serves as an aperture portion for the nanopore. The upper aperture 65 functions to limit the number of nanochannel units 23 constituting the nanochannel 22 connected to the nanopore (the number of nanochannel units constituting the nanochannel connected to the independent nanopore in this example) to one.

In the third configuration of Comparative Example shown in FIG. 6(c), a base pore 66 having upper-surface and lower-surface aperture portions of 50 nm was formed in the base material 11. In this configuration, there is no nanopore that functions to limit the translocation of a nucleic acid strand through the nucleic acid strand translocation pathway to only one molecule of nucleic acid strand.

The step of forming a nanopore of 2.5 nm diameter in the base material 11 was performed with a scanning transmission electron microscope (STEM; HD2700 available from Hitachi High-Technologies) under an acceleration voltage of 200 kV. For example, in the case of the device of the first configuration, the upper aperture 65 was formed in the upper SiN layer of the base material 11, and the SiO₂ layer 62 was etched using the upper aperture 65 as a mask. This was followed by irradiation of the lower SiN layer with a focused electron beam to form the nanopore (lower aperture 64). The pore size was adjusted by varying the electron beam irradiation time. The progress of aperture formation was confirmed by observing a bright-field image obtained by using the STEM used for the formation process.

PEO₁₁₄-b-PMA(Az)₃₄ was deposited on the surface of the base material 11 of the device substrate after forming the aperture using the foregoing procedure. First, PEO₁₁₄-b-PMA(Az)₃₄ was dissolved in toluene in a concentration of 1.5 weight %. The resulting solution was spin coated on a surface of the device substrate in a thickness of about 50 nm. The thickness was adjusted by varying the rotation speed of spin coating. The desired thickness was obtained by performing the spin coating process at a rotation speed of about 3,000 rpm.

The resulting sample was heat annealed in a vacuum oven to cause the PEO₁₁₄-b-PMA(Az)₃₄ thin membrane to self-assemble, and form a microphase-separated structure of the block copolymer. First, the sample was left unattended for 1 hour under 140° C. heated conditions. The heated sample was then cooled to 90° C. to allow a phase transition in PMA(Az)₃₄ from isotropic phase to smectic liquid phase. The cooled sample was allowed to stand in this state for 3 hours, and this was followed by natural cooling. The heat annealing completed the self-assembly of the block copolymer.

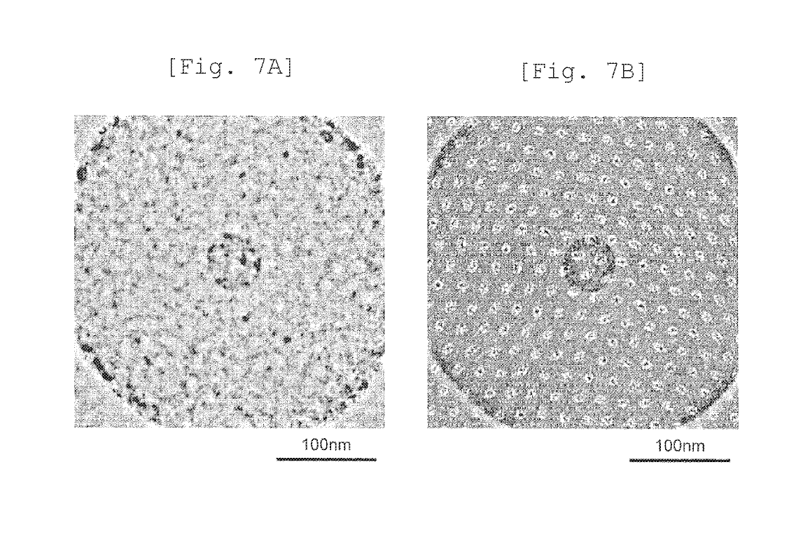

The structure of the nucleic acid transport controlling device was observed by STEM, and the arrangement of the base pore and individual upright cylinders was confirmed. FIG. 7(b) shows an example of a STEM image obtained for the nucleic acid transport controlling device of Example having the first configuration shown in FIG. 6(a). It can be seen from the STEM image that the nanochannel units 23 of a cylindrical PEO structure were hexagonally arranged throughout the device surface, including above the upper aperture 65 of 50 nm diameter. Three PEO cylinders were observed above the upper aperture 65, and four to five PEO cylinders were observed in a region around the upper aperture 65. At the magnification and the contrast used to obtain the STEM image shown in FIG. 7(b), it was riot possible to observe the lower aperture 64 that serves as an aperture portion for the nanopore. However, the presence of the lower aperture 64 was confirmed under different STEM observation conditions.

STEM observation was also performed for the nucleic acid transport controlling devices of Comparative Examples having the second and third configurations, using the same procedure.

In the second configuration shown in FIG. 6(b), the PEO cylinder, and the upper aperture 65 serving as an aperture portion for the nanopore were observed in 1:1 correspondence.

In the third configuration shown in FIG. 6(c), three PEO cylinders were observed above the base pore 66 having an aperture portion of 50 nm diameter, and four to six PEO cylinders were observed in a region around the aperture portion of the base pore 66.

### (3) Evaluation of Nucleic Acid Strand Transport by Nucleic Acid Transport Controlling Device

The ion current that passes through the nucleic acid transport controlling devices of Example (first configuration) and Comparative Examples (second and third configurations) produced in the manner described above was evaluated for behavior and nucleic acid transport.

The evaluation result for the nucleic acid transport controlling device of Example having the first configuration is described first. As described above, in the first configuration, the nanochannel 22 has a structure in which the PEO cylinders representing the independent nanochannel units 23 (three PEO cylinders disposed above a central portion of the upper aperture 65, and four to five PEO cylinders disposed in a region around the upper aperture 65) are disposed parallel to each other. The individual PEO cylinders and the nanopore are connected to each other via the nucleic acid strand aligning portion, which is a space formed by the upper aperture 65 of the base pore.

The nucleic acid transport controlling device having the first configuration was installed in a flow cell made of acrylic resin. The flow cell had solution cells (90 μl volume) on the both sides of the nucleic acid transport controlling device. Flow channels for introducing liquid were provided inside the solution cells. An Ag/AgCl electrode was installed in each solution cell.

A buffer solution was introduced to the solution cells. A mixed solution of 1 M KCl, 10 mM Tris-HCl, and 1 mM EDTA was used as the buffer after adjusting the pH to 7.5.

A voltage was applied between the electrodes using a patch clamp amplifier (Axopatch 200B, available from Axon Instruments), and changes in the ion current passed between the electrodes were measured over time. Signals were recorded in digital at a sampling frequency of 50 kHz using an A/D converter (NI USB-6281, available from National Instruments), after removing high-frequency components with a low-pass filter (cutoff frequency of 5 kHz). The measured ion current amount I under varying voltages V of −100 mV to +100 mV between the electrodes showed linear V-I characteristics.

A 1 nM nucleic acid sample dissolved in the buffer solution was introduced into one of the solution cells through the flow channel. The buffer solution was introduced into the other solution cell. A single-stranded DNA (ssPolyA, base length 1.2 kb, polydeoxyadenylic acid) was used as a nucleic acid sample. A stable, constant ion current was observed upon applying a 100 mV potential between the electrodes, as with the case of when only the buffer solution was introduced to the both cells. An event where the constant ion current showed a spiked current drop was observed at a frequency of about 1 event per second. This event is due to the ion current being blocked by the translocation of the ssPolyA chain through the nanopore present in the translocation pathway of the nucleic acid transport controlling device.

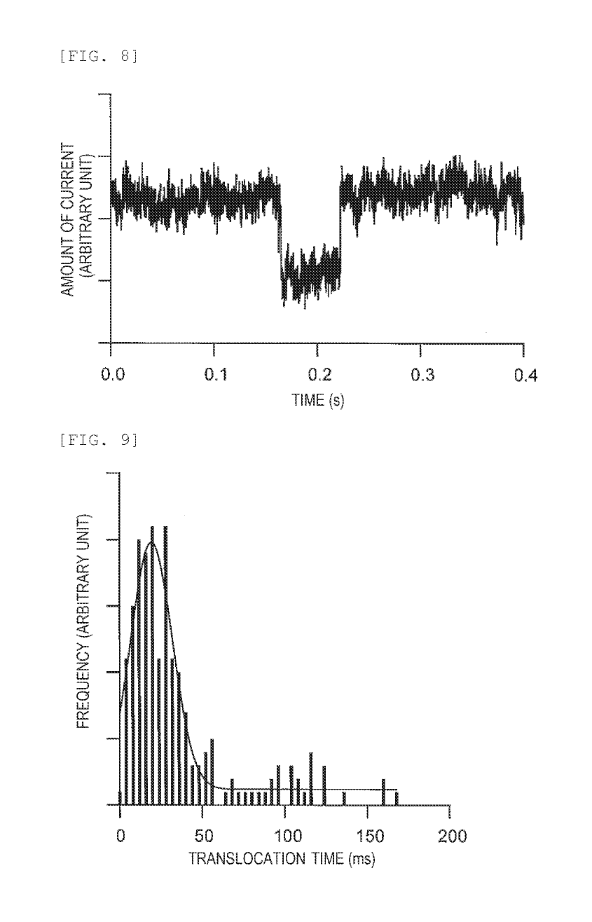

FIG. 8 represents the result of an ion current spike measurement performed at higher time resolution. It was found that spiked current changes occur as continuous rectangular waveforms of a certain blocked current. The duration of individual spikes was evaluated from similar measurement results, and the time needed for the ssPolyA chain to pass through the translocation pathway was measured. FIG. 9 represents a distribution obtained after the duration measurements of large numbers of spikes. As can be seen in FIG. 9, the spike duration had a normal distribution. The duration at the maximum frequency was calculated to be 19 msec. This led to the finding that the time needed for one molecule of ssPolyA chain to pass through the translocation pathway of the nucleic acid transport controlling device was, on average, 19 msec. Since the ssPolyA chain used had a base length of 1200, the translocation time per base was, on average, 16 μsec/base.

As a control, a nucleic acid transport controlling device was prepared by forming a single micropore of 2.5 nm diameter in a SiN thin membrane by STEM. The control nucleic acid transport controlling device had only the base pore (solid state pore), and did not have the block copolymer thin membrane layer. The control nucleic acid transport controlling device was evaluated for nucleic acid strand transport using the same method described above. The translocation time of ssPolyA chain in the control nucleic acid transport controlling device was, on average, 0.01 μsec/base.

It was found from these results that a sufficient amount of stable and constant ion current can be passed, and high-accuracy measurement of block behavior is possible with the first configuration, specifically, with the nucleic acid transport controlling device of Example in which the nucleic acid strand translocation pathway has a single multipath nanochannel per nanopore that allows passage of only one molecule of nucleic acid strand. It was also found that the transport speed of a single-stranded nucleic acid can be greatly reduced with the nucleic acid transport controlling device of Example, as compared to the control nucleic acid transport controlling device having only the solid state pore.

The nucleic acid transport controlling device of Comparative Example having the second configuration was evaluated for nucleic acid strand transport, using the same procedure described above. In the structure of this configuration, the PEO cylinder and the nanopore were disposed in 1:1 correspondence. Specifically, the nanochannel constituting the nucleic acid strand translocation pathway is a single path.

The nucleic acid transport controlling device of Comparative Example was installed in a flow cell, and a buffer solution was introduced into the both solution cells. The measured ion current under an applied potential between the electrodes was about 1/10 of the observed current amount in the nucleic acid transport controlling device of Example having the first configuration. In the observation of ion current I changes under varying voltages V, the V-I characteristics were not straight, but had a shape with the figure of S. Hysteresis was observed in a measurement of current value I performed by sweeping the voltage V.

Time-course changes in the behavior of ion current were measured with a ssPolyA chain introduced to one of the solution cells. A spike that appeared to be due to the translocation of the ssPolyA chain through the nanopore was observed. However, the amount of current change was much smaller than in the nucleic acid transport controlling device of Example having the first configuration, and the S/N ratio against the base noise of constant ion current was insufficient. The translocation time of ssPolyA chain based on spike duration was calculated to be 18 μsec/base, about the same as that obtained with the nucleic acid transport controlling device of Example having the first configuration.

It was found from these results that the effect that slows transport of a single-stranded nucleic acid was sufficient in the nucleic acid transport controlling device of Comparative Example having the second configuration, specifically in the nucleic acid transport controlling device of Comparative Example in which the nucleic acid strand translocation pathway had a single single-path nanochannel per nanopore. However, it was difficult with the nucleic acid transport controlling device of Comparative Example to obtain the S/N ratio of ion current amount and signal required to determine the nucleotide sequence of the nucleic acid strand with the blocked current method.

The nucleic acid transport controlling device of Comparative Example having the third configuration was evaluated for nucleic acid strand transport, using the same procedure described above. In the structure of this configuration, three PEO cylinders are disposed above the base pore, and four to six PEO cylinders are disposed in a region around the aperture portion of the base pore. Specifically, there is no nanopore that can limit the translocation of a nucleic acid strand through the nucleic acid strand translocation pathway to one molecule.

The nucleic acid transport controlling device of Comparative Example was installed in a flow cell, and a buffer solution was introduced into the both solution cells. The observed ion current I changes under varying voltages V of the applied potential between the electrodes had the same linear V-I characteristics observed in the nucleic acid transport controlling device of Example having the first configuration. The absolute value of ion current I was about 10 times that obtained in the nucleic acid transport controlling device of Example.

Time-course changes in the behavior of ion current were measured with a ssPolyA chain introduced to one of the solution cells. In the nucleic acid transport controlling device of this Comparative Example, the clear spike event observed in the nucleic acid transport controlling device of Example having the first configuration, and in the nucleic acid transport controlling device of Comparative Example having the second configuration was not observed. This result is probably due to the nucleic acid transport controlling device of Comparative Example lacking the nanopore that limits the ion current in the translocation of the ssPolyA chain through the nucleic acid strand translocation pathway. It was found from these results that the translocation event of one molecule of single-stranded nucleic acid cannot be evaluated in the third configuration, specifically in the nucleic acid transport controlling device of Comparative Example in which the nanopore is absent, and in which the nucleic acid strand translocation pathway has a nanochannel formed by a plurality of nanochannel units.

These results led to the finding that, in the case of the nucleic acid transport controlling device including the nanochannel having an upright cylindrical structure, the great reduction of the transport speed of a nucleic acid strand achieved while maintaining the ion current characteristics that enable reading the nucleotide sequence with the blocked current method is possible only with the nucleic acid transport controlling device of Example having the first configuration.

### Production Example 2: Fabrication of Nucleic Acid Transport Controlling Device Having Nanochannel of Random Channel Structure

In this Production Example, Example of the nucleic acid transport controlling device of the present invention using the nanochannel having the random channel structure will be described with reference to FIG. 5 to FIG. 7, along with a corresponding Comparative Example.

### (1) Fabrication of Nucleic Acid Transport Controlling Device

In this Production Example, nucleic acid transport controlling devices of two different configurations were fabricated, as schematically represented in the cross sectional structures shown in FIG. 6(d) and (f). The fourth configuration shown in FIG. 6(d) represents Example of the nucleic acid transport controlling device of the present invention. The sixth configuration shown in FIG. 6(f) represents Comparative Example for the fourth configuration of Example. The nucleic acid transport controlling device having the fourth configuration used the same device substrate used in the nucleic acid transport controlling device of the first configuration, and the nucleic acid transport controlling device having the sixth configuration used the same device substrate used in the nucleic acid transport controlling device of the third configuration.

In the fourth configuration of Example shown in FIG. 6 (d), the upper aperture 65 of the base pore formed in the upper surface of the base material 11 had a diameter of 50 nm, and the lower aperture 64 in communication with the upper aperture 65 of the base material 11 had a diameter of 2.5 nm. In this configuration, the lower aperture 64 serves as an aperture portion for the nanopore. The upper aperture 65 serves as an aperture portion for the base pore. The base pore having the upper aperture 65 serves as a nucleic acid strand aligning portion by which the number of the terminal aperture portions of the nanochannel 22 connected to the nanopore is limited within a predetermined range.

In the sixth configuration of Comparative Example shown in FIG. 6(f), a base pore 66 having upper-surface and lower-surface aperture portions of 50 nm was formed in the base material 11. In this configuration, there is no nanopore that functions to limit the translocation of a nucleic acid strand through the nucleic acid strand translocation pathway to only one molecule of nucleic acid strand.

The nanopore of the nucleic acid transport controlling device of Example having the fourth configuration, and the base pore of the nucleic acid transport controlling device of Comparative Example having the sixth configuration were formed by using the same STEM process performed in Production Example 1. Thereafter, a PEO₁₁₄-b-PMA(Az)₃₄ membrane of about 50 nm thickness was deposited on the surface of the base material 11 of the device substrate having the aperture formed therein, using the same spin coating process performed in Production Example 1. The resulting as-spun sample was evaluated in this state without heat annealing, as follows.

The structure of the nucleic acid transport controlling device was observed by STEM, and the arrangement of the base pore and the random channel was confirmed. FIG. 7(a) shows an example of a STEM image obtained for the nucleic acid transport controlling device of Example having the fourth configuration shown in FIG. 6(d). It can be seen from the STEM image that the thin membrane having the nanochannel 22 of a random PEO channel structure was formed throughout the device surface, including above the upper aperture 65 of 50 nm diameter. About four apertures of random channel 22 were observed above the upper aperture 65. At the magnification and the contrast used to obtain the STEM image shown in FIG. 7(a), it was not possible to observe the lower aperture 64 that serves as an aperture portion for the nanopore. However, the presence of the lower aperture 64 was confirmed under different STEM observation conditions.

STEM observation was also performed for the nucleic acid transport controlling device of Comparative Example having the sixth configuration shown in FIG. 6(f), using the same procedure. About four apertures of random channel were observed above the base pore 66 of 50 nm diameter.

### (2) Evaluation of Nucleic Acid Strand Transport by Nucleic Acid Transport Controlling Device

The ion current that passes through the nucleic acid transport controlling devices of Example (fourth configuration) and Comparative Example (sixth configuration) produced in the mariner described above was evaluated for behavior and nucleic acid transport.

The evaluation result for the nucleic acid transport controlling device of Example having the fourth configuration is described first. As described above, in the fourth configuration, the nanochannel 22 has a random channel structure. The nanochannel 22 of a random channel structure has a co-continuous structure of a plurality of continuous hydrophilic PEO paths. The terminal apertures of the random channel 22, and the nanopore are connected to each other via the nucleic acid strand aligning portion, which is a space formed by the upper aperture 65 of the base pore. Because of this structure, the nucleic acid strand translocation pathway of Example has a single multipath nanochannel per nanopore.

The nucleic acid transport controlling device of Example having the fourth configuration was installed in a flow cell, and a buffer solution was introduced into the both solution cells, as in Production Example 1. Ion current I changes were observed under varying voltages V of the applied potential between the electrodes, using the same procedure used in Production Example 1. The V-I characteristics were linear.

Time-course changes in the behavior of ion current were measured with a ssPolyA chain introduced to one of the solution cells, using the same procedure used in Production Example 1. A stable, constant ion current was observed, as with the case of when only the buffer solution was introduced to the both solution cells. An event where the constant ion current showed a spiked current drop was observed. An ion current spike measurement conduced at higher time resolution revealed that spiked current changes occur as continuous rectangular waveforms of a certain blocked current, as in the result observed for the nucleic acid transport controlling device of Example having the first configuration.

The duration of individual spikes was evaluated using the same procedure used in Production Example 1, and the time needed for the ssPolyA chain to pass through the translocation pathway was measured. The spike duration had a normal distribution. The duration at the maximum frequency was calculated to be 22 msec. This led to the finding that the time needed for one molecule of ssPolyA chain to pass through the translocation pathway of the nucleic acid transport controlling device was, on average, 22 msec. Since the ssPolyA chain used had a base length of 1200, the translocation time per base was, on average, 18 μsec/base.

It was found from these results that the transport speed of a single-stranded nucleic acid can be greatly reduced while maintaining a sufficient amount of stable ion current with the fourth configuration, specifically, with the nucleic acid transport controlling device of Example in which the nucleic acid strand translocation pathway has a single multipath nanochannel per nanopore that allows passage of only one molecule of nucleic acid strand.

The nucleic acid transport controlling device of Comparative Example having the sixth configuration was evaluated for nucleic acid strand transport, using the same procedure described above. In the structure of this configuration, the nanochannel 22 of a random channel structure having a plurality of terminal apertures is disposed above the base pore 66. Specifically, there is no nanopore that can limit the translocation of a nucleic acid strand through the nucleic acid strand translocation pathway to one molecule.

The nucleic acid transport controlling device of Comparative Example having the sixth configuration was installed in a flow cell, and a buffer solution was introduced into the both solution cells, as in Production Example 1. Ion current I changes were observed under varying voltages V of the applied potential between the electrodes, using the same procedure used in Production Example 1. The V-I characteristics were linear, as in the nucleic acid transport controlling device of Example having the first configuration. The absolute value of ion current I was about 10 times that obtained in the nucleic acid transport controlling device having the first configuration.

Time-course changes in the behavior of ion current were measured with a ssPolyA chain introduced to one of the solution cells, using the same procedure used in Production Example 1. In the nucleic acid transport controlling device of this Comparative Example, the clear spike event observed in the nucleic acid transport controlling device of Example having the first configuration, the nucleic acid transport controlling device of Comparative Example having the second configuration, and the nucleic acid transport controlling device of Example having the fourth configuration was not observed. This result is probably due to the nucleic acid transport controlling device of Comparative Example lacking the nanopore that limits the ion current in the translocation of the ssPolyA chain through the nucleic acid strand translocation pathway. It was found from these results that the translocation event of one molecule of single-stranded nucleic acid, cannot be evaluated in the sixth configuration, specifically in the nucleic acid transport controlling device of Comparative Example in which the nanopore is absent, and in which the nucleic acid strand translocation pathway has a nanochannel having a random channel structure.

These results led to the finding that, in the case of the nucleic acid transport controlling device including the nanochannel having a random channel structure, the great reduction of the transport speed of a nucleic acid strand achieved while maintaining the ion current characteristics that enable reading the nucleotide sequence with the blocked current method is possible only with the nucleic acid transport controlling device of Example having the fourth configuration.

### Production Example 3: Fabrication of Nucleic Acid Transport Controlling Device Having Nanochannel of Random Channel Structure Using Different Process

In this Production Example, another Example of the nucleic acid transport controlling device of the present invention using the nanochannel having the random channel structure will be described with reference to FIG. 6.

### (1) Fabrication of Nucleic Acid Transport Controlling Device

In this Production Example, a nucleic acid transport controlling device of the configuration schematically represented in the cross sectional structure shown in FIG. 6(e) was fabricated. The fifth configuration shown in FIG. 6(e) represents Example of the nucleic acid transport controlling device of the present invention. The feature of the method used in this Production Example lies in the step of forming the block copolymer thin membrane 20 on the upper surface of a base material 11 having no aperture, the step of forming the nanochannel 22 in the block copolymer thin membrane using an optionally performed additional process, such as heat annealing, and the step of forming the nanopore 13.

In this Production Example, a nucleic acid transport controlling device of a configuration having the structure schematically represented in FIG. 6(e) was fabricated. First, the base material 11 was formed on the upper surface of a Si wafer provided as the support substrate 12. A window was provided in the support substrate 12 by anisotropic etching of the Si wafer with KOH, and a lower aperture 64 was formed in a lower SiN membrane 63 and a SiO₂ layer 62, using a photolithography process. It should be noted here that the upper aperture 65, which becomes the nanopore, is not formed at this stage.

Thereafter, a PEO₁₁₄-b-PMA(Az)₃₄ membrane of about 50 nm thickness was formed on the surface of the base material of the device substrate having the aperture formed therein, using the same spin coating process performed in Production Example 1. The resulting as-spun sample was used in the subsequent steps in this state, without heat annealing, as follows.

The as-spun sample obtained in the foregoing process was installed in the flow cell used in Production Example 1. A 1 M KCl aqueous solution was then introduced into the both solution cells after adjusting the pH to 10.0. Apulsed voltage was continuously applied between the electrodes, and an upper aperture 65 that serves as an aperture portion for the nanopore was formed in the upper SiN membrane 61. In this step, the current amount passing between the electrodes under applied voltage was measured, and the nanopore ot the desired diameter (1.5 nm in this Example) was formed.

As shown in FIG. 6(e), the terminal aperture of the PEO random, channel of a random, channel structure, and the nanopore need to be disposed in 1:1 correspondence in the nucleic acid transport controlling device of Example having the fifth, configuration. For this reason, the terminal aperture of the nanochannel needs to be accurately aligned one-to-one with the previously formed nanopore in the method of production used, in Production Examples 1 and 2, specifically in the method in which the step of forming a nanopore in the device substrate is followed by the step of forming a block copolymer thin membrane, and the step of forming a nanochannel through self-assembly of the block copolymer. In contrast, in the method of production used in this Production Example, the nanopore is formed at the path terminal of the PEO random channel that passes current under applied pulsed voltage. Accordingly, the terminal aperture of the nanochannel aligns itself with the previously formed nanopore in 1:1 correspondence. That is, the method of production used in this Production Example does not require aligning the terminal aperture of the nanochannel with the nanopore, and can produce the nucleic acid transport controlling device of the present invention with ease.

### (2) Evaluation of Nucleic Acid Strand Transport by Nucleic Acid Transport Controlling Device

The ion current that passes through the nucleic acid transport controlling device of Example produced in the manner described above was evaluated for behavior and nucleic acid transport.

As described above, the nanochannel 22 has a random channel structure in the fifth configuration. The nanochannel 22 of a random channel structure has a co-continuous structure of a plurality of continuous hydrophilic PEO paths. The terminal aperture of the nanochannel 22 having a random channel structure is directly connected to the nanopore, one-to-one. Because of this structure, the nucleic acid strand translocation pathway of this Example has a single multipath nanochannel per nanopore.

The nucleic acid transport controlling device of Example having the fifth configuration was installed in a flow cell, and a buffer solution was introduced into the both solution cells, as in Production Example 1. Ion current I changes were observed under varying voltages V of the applied potential between the electrodes, using the same procedure used in Production Example 1. The V-I characteristics were linear.

Time-course changes in the behavior of ion current were measured with a ssPolyA chain introduced to one of the solution cells, using the same procedure used in Production Example 1. A stable, constant ion current was observed, as with the case of when only the buffer solution was introduced to the both solution cells. An event where the constant ion current showed a spiked current drop was observed. An ion current spike measurement conduced at higher time resolution revealed that spiked current changes occur as continuous rectangular waveforms of a certain blocked current, as in the result observed for the nucleic acid transport controlling device of Example having the first configuration.

The duration of individual spikes was evaluated using the same procedure used in Production Example 1, and the time needed for the ssPolyA chain to pass through the translocation pathway was measured. The spike duration had a normal distribution. The duration at the maximum frequency was calculated to be 22 msec. This led to the finding that the time needed for one molecule of ssPolyA chain to pass through the translocation pathway of the nucleic acid transport controlling device was, on average, 22 msec. Since the ssPolyA chain used had a base length of 1200, the translocation time per base was, on average, 18 μsec/base.

The experiment of Production Example 1 was conducted using the same procedure, except that the 1.2 kb ssPolyA chain was replaced with a shorter single-stranded DNA (ssPolyA(60), base length 60 b, polydeoxyadenylic acid). The ssPolyA(60) chain was then evaluated for transport behavior. A stable, constant ion current was observed as with the case of using the 1.2 kb ssPolyA chain. An event where the constant ion current snowed a spiked current drop was observed. In the experiment conducted with the ssPolyA(60) chain, the frequency of this event was higher than in the experiment conducted with the 1.2 kb ssPolyA chain.

An ion current spike measurement conduced at higher time resolution revealed that spiked current changes occur as continuous rectangular waveforms of a certain blocked current. The duration of individual spikes was evaluated from similar measurement results, and the time needed for the ssPolyA(60) chain to pass through the translocation pathway was measured. The spike duration had a normal distribution. The duration at the maximum frequency was calculated to be 0.8 msec. This led to the finding that the time needed for one molecule of ssPolyA(60) chain to pass through the translocation pathway of the nucleic acid transport controlling device was, on average, 0.85 msec. Since the ssPolyA(60) chain used had a base length of 60, the translocation time per base was, on average, 14 μsec/base.

It was found from these results that the transport speed of a single-stranded nucleic acid can be greatly reduced while maintaining a constant ion current with the fifth configuration, specifically, with the nucleic acid transport controlling device of Example in which the nucleic acid strand translocation pathway has a single multipath nanochannel per nanopore that allows passage of only one molecule of nucleic acid strand. It was also found that transport of a shorter-length single-stranded nucleic acid also can be controlled with the nucleic acid transport controlling device of Example having the fifth configuration. This is considered to be due to the structure of the nucleic acid transport controlling device of Example of the fifth configuration in which the nanochannel of a random channel structure of PEO chains having the transport slowing effect is directly connected to the nanopore.

The present invention is not limited to the examples described above, and includes many variations. For example, the foregoing examples described to help illustrate the present invention are not necessarily required to include all the configurations described above. It is also possible to add other configuration, or delete and/or replace a part of the configuration of each example.

All publications, patents, and patent application cited in this specification are incorporated herein by reference in its entirety.

### REFERENCE SIGNS LIST

10: Nucleic acid transport controlling device

11: Base material

12: Support substrate

13: Nanopore

14: Translocation pathway

15: Base pore

20: Block copolymer thin membrane

21: Hydrophobic matrix

22: Hydrophilic nanochannel

23: Hydrophilic nanochannel unit

30: Solution cell

31: Nucleic acid strand

32: Electrode

33: Electrolyte solution

34: Power supply

35: Ammeter

40: Block copolymer

41: Hydrophobic polymer chain

42: Hydrophilic polymer chain

43: Linkage point

61: SiN thin membrane

62: SiO₂ thin membrane

63: SiN thin membrane

64: Lower aperture

65: Upper aperture

66: Base pore

1. A nucleic acid transport controlling device comprising a nucleic acid strand translocation pathway,
wherein the nucleic acid strand translocation pathway includes one or more multipath Nano channels per nanopore that allows passage of only one molecule of nucleic acid strand,
wherein the Nano channels have a microphase-separated structure of a block copolymer of a hydrophobic polymer chain and a hydrophilic polymer chain, and
wherein the Nano channels contain the hydrophilic polymer chain of the block copolymer as a main component.

2. The nucleic acid transport controlling device according to claim 1, wherein the nucleic acid strand translocation pathway includes a single multipath nanochannel per nanopore that allows passage of only one molecule of nucleic acid strand.

3. The nucleic acid transport controlling device according to claim 1, wherein the nanopore and the nanochannels are disposed in contact with each other, or by being separated from each other.

4. The nucleic acid transport controlling device according to claim 1, further comprising:
an insulating base material; and
a thin membrane directly or indirectly disposed above the insulating base material and containing the block copolymer,
wherein the insulating base material includes the nanopore, and
wherein the thin membrane includes the Nano channels, and a matrix disposed around the Nano channels.

5. The nucleic acid transport controlling device according to claim 1, wherein the nanochannels have a branched structure.

6. The nucleic acid transport controlling device according to claim 1, wherein the nanochannels and the matrix have a co-continuous structure.

7. The nucleic acid transport controlling device according to claim 3, wherein the nanopore and the nanochannels are disposed by being separated from each other, and wherein the nanochannels have a cylindrical structure.

8. The nucleic acid transport controlling device according to claim 1, wherein the hydrophilic polymer chain includes polyethylene oxide, polylactic acid, polyhydroxyalkylmethacrylate, polyacrylamide, polyacrylic acid, a polyamino acid, or a nucleic acid.

9. The nucleic acid transport controlling device according to claim 1, wherein the hydrophobic polymer chain has a liquid-crystalline side chain.

10. The nucleic acid transport controlling device according to claim 9, wherein the hydrophobic polymer chain has a structure in which the alkyl moiety of the polyalkylmethacrylateis partially or completely substituted with theliquid-crystallinechain.

11. A nucleic acid transport controlling device comprising a nucleic acid strand translocation pathway,
wherein the nucleic acid strand translocation pathway includes one or more multipath Nano channels per nanopore that allows passage of only one molecule of nucleic acid strand,
wherein the nucleic acid transport controlling device includes an insulating base material having one or more of the nanopore, and a thin membrane directly or indirectly disposed above the insulating base material,
wherein the thin membrane includes one or more of the nanochannels, and a matrix disposed around the Nano channels, and
wherein the nanochannels are packed with a hydrophilic polymer chain immobilized at the interface between the nanochannels and the matrix.

12. A method for manufacturing a nucleic acid transport controlling device that includes a nucleic acid strand translocation pathway that includes one or more multipath Nano channels per nanopore that allows passage of only one molecule of nucleic acid strand,
the method comprising the steps of:
forming the nanopore in the insulating base material;
forming a thin membrane of a block copolymer of a hydrophobic polymer chain and a hydrophilic polymer chain above the insulating base material; and
causing the block copolymer to self-assemble, and form a Nano channel having a microphase-separated structure of the block copolymer and containing the hydrophilic polymer chain as a main component.

13. The method according to claim 12, wherein the step of forming the nanopore is performed after the step of forming the Nano channel.

14. A nucleic acid sequencing apparatus comprising:
the nucleic acid transport controlling device of claim 1;
two solution cells that are in communication with each other via the nucleic acid strand translocation pathway of the nucleic acid transport controlling device; and
an electrode provided for each of the two solution cells to apply a voltage between the solution cells.

15. The nucleic acid sequencing apparatus according to claim 14, wherein the nucleic acid transport controlling device has a plurality of nucleic acid strand translocation pathways that are disposed parallel to each other.

16. The nucleic acid sequencing apparatus according to 14, further comprising a device for measuring a time-course change of a current amount passed between the electrodes.
